(19) United States
(12) Patent Application Publication
(10) Pub. No.: US 2024/0093181 A1
(43) Pub. Date: Mar. 21, 2024

(54) METHODS FOR ISOLATING AND/OR OBTAINING CAPTURED POLYNUCLEOTIDE FRAGMENTS

(71) Applicants: Cosimo JANN, Heidelberg (DE); S. Andreas JOHANSSON, Heidelberg (DE); Aaron N. BROOKS, Heidelberg (DE); Amanda HUGHES, Heidelberg (DE); Lars M. STEINMETZ, Heidelberg (DE)

(72) Inventors: Cosimo JANN, Heidelberg (DE); S. Andreas JOHANSSON, Heidelberg (DE); Aaron N. BROOKS, Heidelberg (DE); Amanda HUGHES, Heidelberg (DE); Lars M. STEINMETZ, Heidelberg (DE)

(21) Appl. No.: 18/501,530

(22) Filed: Nov. 3, 2023

Related U.S. Application Data
(63) Continuation of application No. PCT/EP2022/061768, filed on May 3, 2022.

(30) Foreign Application Priority Data
May 3, 2021 (EP) 21171858.0

(51) Int. Cl. C12N 15/10

(52) U.S. Cl. CPC C12N 15/1082; C12N 15/1058; C12N 15/1065

(57) ABSTRACT

The present invention pertains to methods for obtaining captured polynucleotide fragments. Further provided are methods for obtaining rearranged captured polynucleotide fragments. The present invention also relates to methods for the identification of at least one compound, at least one protein, and/or at least one secondary metabolite having biological activity. Kits for performing the above methods, methods of treatment using the compounds as screened with the methods for identification of at least one compound having biological activity, as well as pharmaceutical compositions thereof, are also provided.

### FIELD OF THE INVENTION

The present invention pertains to methods for obtaining captured polynucleotide fragments. Further provided are methods for obtaining rearranged captured polynucleotide fragments. The present invention also relates to methods for the identification of at least one compound, at least one protein, and/or at least one secondary metabolite having biological activity. Kits for performing the above methods, methods of treatment using the compounds as screened with the methods for identification of at least one compound having biological activity, as well as pharmaceutical compositions thereof, are also provided.

### DESCRIPTION

An enormous amount of effort is being made by scientists worldwide to identify compounds having biological activity. Such compounds can be used to treat diseases, improve life quality, and/or protect the environment. The discovery of novel compounds or proteins having a desired biological activity lies in the ability to access and study any kind of molecule derived from any kind of organism and/or sample, such as those present in natural, environmental samples. Most previous methods for discovering functional molecules derived from different kinds of samples, such as environmental samples, depended on cultivation of organisms, e.g. by growing bacteria or fungi in the laboratory, and partial restriction digest of nucleic acids derived from said organisms to obtain nucleic acid fragments. Such methods are done by destroying all cells (e.g. in a soil sample), purifying DNA, performing a partial restriction digest and ligating fragments containing two restriction sites into e.g. bacterial artificial chromosomes (BAC) or yeast artificial chromosomes (YAC), which are subsequently subjected to functional screenings.

A huge problem of such methods is that only cultivatable organisms can be analyzed for their ability to produce compounds and/or proteins potentially having a biological activity. However, it is well-established that many, perhaps a vast majority of, organisms in certain environmental samples cannot be readily cultivated with known isolation and incubation methods, such as a large number of bacteria and fungi derived from natural environments. In soil, estimates are that 80 to 99% of the microorganisms remain so far unidentified and non-cultivatable.

Therefore, it was so far not possible to screen a huge variety of molecules and/or compounds derived from environmental samples, such as samples derived from soil or seawater, for their biological activity, and there is an urgent need for methods that allow analysis of any kind of polynucleotide molecule produced by non-cultivatable organisms that might have a biological activity, such as an enzymatic activity. The ability to capture, analyze, and sequence polynucleotide molecules from so far uncharacterized organisms is therefore of great interest. Moreover, there is a huge need for identifying so far uncharacterized organisms. Current methods for identification of new organisms are typically based on sequencing, which requires the presence of a known nucleic acid sequence. However, such knowledge is not always available.

WO2012055408 (A1) discloses methods for specific capture of single stranded target polynucleotide molecules by a complementary probe comprising one or more intercalator molecules.

WO2004104179 (A2) pertains to methods and reagents for isolating polynucleotide molecules from uncultured microbial cells in an environmental sample, the method comprising obtaining uncultured microbial cells from the sample, dispersing the cells from each other and from other components in the sample, purifying the dispersed cells via discontinuous gradient centrifugation wherein the cells are collected in an interface of the gradient, embedding the cells in agarose gel, to produce agarose gel blocks containing the cells, and lysing the cells within the agarose gel blocks, thereby releasing high molecular weight polynucleotide molecules from the cells.

WO2014093676 (Al) provides compositions, methods, systems, and devices for polynucleotide processing, which may be useful for a variety of applications, including polynucleotide sequencing.

In view of the above, there is a continuing need in the art to obtain captured polynucleotide fragments from any kind of sample. There is also a continuing need in the art to identify new organisms, including non-cultivatable organisms. There is a further need to capture low abundant, random DNA. In addition, it is desired to have a technique that allows a cheap and easy way to synthesize entire genetic pathways. A preferred tool for capturing polynucleotide fragments will have several fundamental characteristics. It should be universally applicable to all different kinds of molecules and/or organisms derived from any kind of sample, and should be easy and fast to use. The tool should further allow the subsequent analysis of the biological activity of any kind of molecule produced from said captured polynucleotide fragments.

The invention solves the above problems by providing a novel method for obtaining a captured polynucleotide fragment in a fast, easy-to-use and reliable way. The present invention is based on a novel use of transposases, such as Tn5 transposases, by modifying transposase linker sequences to comprise at least one capture sequence tag (CST), i.e. adaptors comprising at least one capture sequence tag (CST). The invention allows in vitro transposon-mediated fragmenting and tagging of any nucleic acid molecule derived from any kind of sample. The invention further allows combining the above method with a method for identification of at least one compound, at least one protein, and/or at least one secondary metabolite having biological activity.

### BRIEF DESCRIPTION OF THE INVENTION

Generally, and by way of brief description, the main aspects of the present invention can be described as follows:

In a first aspect, the invention pertains to a method for obtaining a captured polynucleotide fragment.

In a second aspect, the invention pertains to a method for obtaining a rearranged captured polynucleotide fragment.

In a third aspect, the invention pertains to a kit for performing a method according to the present invention.

In a fourth aspect, the invention pertains to a polynucleotide fragment library comprising at least one captured polynucleotide fragment.

In a fifth aspect, the invention pertains to a method for identification of at least one compound, at least one protein, and/or at least one secondary metabolite having biological activity

In a sixth aspect, the invention relates to a compound for use in the treatment of a disease.

### DETAILED DESCRIPTION OF THE INVENTION

In the following, the elements of the invention will be described. These elements are listed

with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In the first aspect, the invention pertains to a method for obtaining a captured polynucleotide fragment, the method comprising the steps of:
a) Providing a sample, an acceptor molecule, at least one enzyme, and at least one transposase, mutant transposase or variant transposase, wherein the at least one transposase, mutant transposase or variant transposase is bound to at least two adaptors, and wherein each adaptor comprises at least one capture sequence tag (CST);
b) Optionally, isolating at least one polynucleotide molecule from the sample;
c) Contacting the sample and/or the isolated polynucleotide molecule with the at least one transposase, mutant transposase or variant transposase, thereby generating a polynucleotide fragment, wherein at least one, preferably both ends, of the polynucleotide fragment is tagged with the at least one CST,
d) Contacting the polynucleotide fragment and/or the acceptor molecule with the at least one enzyme, thereby inserting the polynucleotide fragment into the acceptor molecule to obtain the captured polynucleotide fragment.

The inventors surprisingly found that modifying the transposase linker sequence of a transposase to introduce a sequence of interest, e.g. a capture sequence tag (CST) sequence, allows to provide a fast, easy-to-use and reliable method for obtaining captured polynucleotide fragments. Importantly, a transposon-mediated tagmentation reaction can be used to insert a tag sequence at a random location into a polynucleotide, and make a double-stranded cut in the polynucleotide, to yield a polynucleotide fragment appended at one or both ends with at least one tag. For example, a transposon complex can be formed by contacting a polynucleotide with a transposase which is bound to two transposon end sequences each containing at least one tag. The transposon complex can be incubated under conditions that permit a tagmentation reaction to occur. Hence, the present invention is based on a novel use of transposases, such as Tn5 transposases, by modifying transposase linker sequences to comprise at least one CST, i.e. adaptors comprising at least one CST. The invention allows in vitro transposon-mediated fragmenting and tagging (e.g., “tagmentation”) of any nucleic acid molecule derived from any kind of sample, for example an environmental sample derived from soil or seawater.

The method for obtaining a captured polynucleotide fragment according to the present invention is advantageous over state of the art methods. Importantly, partial restriction digests always rely on the presence of restriction sites. However, many genomes may not contain the specific restriction site of a chosen restriction enzyme and can thus not be captured using prior art methods. Additionally or alternatively, many genomes may not contain the specific site of a chosen restriction enzyme in high frequency and these genomes are thus cleaved to small fragments. In order to gain large fragments and to avoid overdigestion, state of the art methods usually use restriction enzymes with rare restriction enzyme recognition sites. However, it is so far not known how common these rare restriction enzyme recognition sites are present in unknown genomes.

A second disadvantage of prior art methods compared to the present method for obtaining a captured polynucleotide fragment is that commonly used methods using partial restriction digests often result in the capture of very large fragments (>100 kb). However, in case a bioactivity can be observed for a protein and/or product of such a large fragment, it is very difficult to identify a single gene or single genetic cluster within the large fragment that is responsible for the measured bioactivity without further genetic engineering. In addition, other genes of this fragment that are expressed can have adverse effects on the expression of a desired gene(s) or the bioactivity of their product, as well as on the viability of the transformed cell. For example, microbes can produce a variety of toxins and/or restriction enzymes that could kill the heterologous host when produced. In such a case, unfortunately no product having a bioactivity can be identified. The present invention overcomes such disadvantages of prior art methods by making use of a transposase-based method, and it also allows to overcome the low efficiency of ligation-based reactions by a transposase-mediated method with higher efficiency.

Preferably, the sample as provided in step a) of the above method for obtaining a captured polynucleotide fragment according to the first aspect of this invention comprises at least one polynucleotide molecule.

The term “at least one” according to the present invention shall include 1, 2, 3, 4, 5, 6, 7, 8, 9, 10, 11, 12, 13, 14, 15, 16, 17, 18, 19, 20, or any other number. For example, the term “at least one” enzyme shall include 1, 2, 3, 4, 5, 6, 7, 8, 9, 10, 11, 12, 13, 14, 15, 16, 17, 18, 19, 20, or any other number of enzymes of. Similarly, the term “at least one” nucleic acid shall include 1, 2, 3, 4, 5, 6, 7, 8, 9, 10, 11, 12, 13, 14, 15, 16, 17, 18, 19, 20, or any other number of nucleic acids.

In a preferred embodiment, the at least one polynucleotide molecule, the polynucleotide fragment, and/or the captured polynucleotide fragment is a functional or non-functional DNA polynucleotide molecule, such as a single-stranded or double-stranded DNA polynucleotide molecule, or a fragment or derivative thereof, for example a cDNA molecule, a DNA aptamer, and/or a protein-coding or non-coding DNA. In one embodiment, the at least one polynucleotide molecule, the polynucleotide fragment, and/or the captured polynucleotide fragment is a functional or non-functional DNA polynucleotide molecule which can be extracted from RNA and converted to cDNA using either hexamers or targeted oligo (dT) primers before capture.

In a preferred embodiment, the contacting in step c) of the method according to the first aspect of the present invention is performed under conditions suitable for tagmentation. The polynucleotide fragment as generated in step c) is preferably a tagmented polynucleotide fragment. As used herein, the term “tagmentation” refers to the modification of DNA by a transposome complex comprising a transposase enzyme and transposon end sequence in which the transposon end sequence further comprises an adaptor sequence. Tagmentation results in the simultaneous fragmentation of the DNA and ligation of the adaptors to the 5′ ends of both strands of duplex fragments.

Another preferred embodiment relates to the above method for obtaining a captured polynucleotide fragment, wherein the transposase is a DDE transposase, a Tn5 transposase, a Tn3 transposase, a Tn7 transposase, a Tn10 transposase, a Tn552 transposase, a Tn903 transposase, a sleeping beauty transposase, a Mu transposase, a MuA transposase, Mosi, Hermes, ProtoRAG, a HUH-like (Y1-/Y2-) transposase, a Y-transposase and/or a S-transposase, such as Tn1549, a Vibhar transposase, such as a Vibhar transposase from Vibrio harveyi, or a mutant or variant thereof, preferably wherein the transposase is Tn5 transposase, or a mutant or variant thereof.

Different transposases use different mechanisms, such as a cut-and-paste process or a replicative process. In the cut-and-paste process, DNA double strand breaks are induced and the transposon is excised from its original location. Then, integration occurs by attack of free 3′-OH groups on a target DNA. On the other hand, in replicative transposition processes, the element is nicked on both ends and integration creates a so-called Shapiro intermediate. Subsequently, this intermediate is resolved by replication, generating a new transposon copy at the target site. Alternatively, some transposases are able to combine features of both cut-and-paste process and replicative process, such as utilizing replication to proceed via excised circular intermediates. Y-transposases and S-transposases are an example for transposases that make use of the cut-and-paste process. Excision of DNA by a Y-transposase or an S-transposase creates a double-stranded circular intermediate with the transposon ends adjoined, with a short stretch (5-7 base pairs) of flanking DNA between the ends enclosed. The donor DNA is simultaneously resealed. Recombination of the transposon with target DNA then leads to integration.

In a particularly preferred embodiment, the transposase is a hyperactive variant of Tn5 transposase, which is characterized by a low integration site preference, a high degree of transposition, and the possibility of loading with adapter sequences that allow DNA fragmentation. According to the present invention, any transposase with such properties can be used for the method for obtaining a captured polynucleotide fragment. While most transposases do not have these properties, they may be engineered towards increasing such beneficial and desired properties, such as hyperactive Sleeping Beauty variants.

In yet another preferred embodiment, the acceptor molecule is a DNA molecule, such as a single-stranded or double-stranded DNA polynucleotide molecule, or a fragment or derivative thereof, preferably wherein the acceptor molecule is a DNA polynucleotide molecule selected from a plasmid, a bacterial artificial chromosome (BAC), a yeast artificial chromosome (YAC), viral DNA, and/or genomic DNA.

A further embodiment relates to the above method for obtaining a captured polynucleotide fragment, wherein the at least one capture sequence tag (CST) is a lox recombination sequence, such as a loxP sequence, preferably a symmetric loxP (symLoxP) sequence, a rox recombination sequence, a restriction enzyme recognition sequence, such as a Typal sequence, and/or a homology sequence.

In some embodiments, the nucleic acid amplification reaction includes utilizing a LoxP/Cre system, in which a LoxP sequence is joined to at least one tag and a Cre recombinase is used to generate a circular molecule having a tag insert.

In another embodiment, the acceptor molecule comprises at least one acceptor nucleic acid sequence, such as an acceptor loxP sequence, preferably an acceptor symLoxP sequence, an acceptor Rox sequence, a restriction enzyme recognition sequence, for example a TypeII sequence, and/or a homology sequence.

A further preferred embodiment pertains to the above method for obtaining a captured polynucleotide fragment, wherein the at least one enzyme is Cre recombinase, Dre recombinase, a restriction enzyme, a ligase, and/or a homology-directed repair (HDR) enzyme. The at least one enzyme can be contained in at least one living cell or at least one cell-free system, or can be a purified enzyme.

In one embodiment, the invention pertains to a method for obtaining a captured polynucleotide fragment, the method comprising the steps of:
a) Providing a sample, an acceptor molecule, at least one enzyme, and at least one transposase, mutant transposase or variant transposase, wherein the at least one transposase, mutant transposase or variant transposase is bound to at least two adaptors, and wherein each adaptor comprises at least one CST;
b) Optionally, isolating at least one polynucleotide molecule from the sample;
c) Contacting the sample and/or the isolated polynucleotide molecule with the at least one transposase, mutant transposase or variant transposase, thereby generating a polynucleotide fragment, wherein at least one, preferably both ends, of the polynucleotide fragment is tagged with at least one CST,
d) Contacting the polynucleotide fragment and/or the acceptor molecule with the at least one enzyme, thereby inserting the polynucleotide fragment into the acceptor molecule to obtain the captured polynucleotide fragment, wherein said method does not comprise the use of a nuclease, preferably wherein the at least one CST is a lox recombination sequence, such as a loxP sequence, preferably a symmetric loxP (symLoxP) sequence, and/or a rox recombination sequence.

In another embodiment, which can be combined with any of the other embodiments, the enzyme provided in step a) is not a nuclease. Accordingly, a method can be preferred wherein the enzyme provided in step a) is not a nuclease. This is particularly advantageous because it allows to overcome the low efficiency of ligation reactions by using other reactions with higher efficiency.

In yet another embodiment, step c) comprises ligating at least one CST to the 5′ end of at least one, preferably both ends, of the at least one polynucleotide fragment.

Further preferred is that the inserting of step d) occurs by recombination, restriction and ligation, and/or homology-directed repair (HDR). After insertion as specified in step d), the at least one captured polynucleotide fragment can be stored and/or amplified, such as stored and/or amplified in form of at least one captured polynucleotide fragment present in a vector.

In a preferred embodiment, the captured polynucleotide fragment has a length of at least 1 nucleotide base, preferably of at least 10 nucleotide bases, more preferably of at least 100 nucleotide bases, even more preferably of at least 10³ nucleotide bases.

In a further embodiment, the captured polynucleotide fragment has a length of between 1 and 10⁸ nucleotide bases, preferably between 10 and 10⁷ nucleotide bases, more preferably between 100 and 10⁶ nucleotide bases, even more preferably between 10³ and 10⁵ nucleotide bases, and most preferably between 5×10³ and 5×10⁴ nucleotide bases.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure. In particularly preferred embodiments of the invention, the term “about” may refer to a deviation of the respective numeric value of a maximum of 20% of the numerical value, however more preferred is 15%, 10%, 5%, even more preferred is 4%, 3%, 2%, and most preferred is 1%.

In a particularly preferred embodiment, the length of the captured polynucleotide fragment depends on the ratio of the at least one transposase, mutant transposase or variant transposase, to the at least one polynucleotide molecule in the sample, and/or the duration of the contacting in step c). The present invention provides thus a method for obtaining a captured polynucleotide fragments, wherein the length distribution of the tagmented polynucleotide molecule fragments is tunable. Importantly, tagmented polynucleotide molecule fragments can be varied from very small tagmented products (<300 bp) to very large fragments (>100 kbp). During tagmentation, the ratio of DNA to transposase and the length of transposase treatment determines the length distribution of tagmented DNA fragments and can be modulated to yield fragments of any defined and/or desired size. This allows obtaining polynucleotide fragments large enough to comprise at least one, or a multitude of genes, or even genes of entire pathways, such as metabolic pathway clusters and/or operons.

A further embodiment pertains to the above method for obtaining a captured polynucleotide fragment, wherein the length of the captured polynucleotide fragment can be (i) increased by decreasing the ratio of the at least one transposase, mutant transposase or variant transposase, to the at least one polynucleotide molecule in the sample, or (ii) decreased by increasing the ratio of the at least one transposase, mutant transposase or variant transposase, to the at least one polynucleotide molecule in the sample.

Another embodiment relates to the above method for obtaining a captured polynucleotide fragment, wherein (i) if the ratio of the at least one transposase, mutant transposase or variant transposase, to the at least one polynucleotide molecule in the sample, is lower compared to a reference value or reference sample, the length of the captured polynucleotide fragment is higher, and/or wherein (ii) if the ratio of the at least one transposase, mutant transposase or variant transposase, to the at least one polynucleotide molecule in the sample, is higher compared to a reference value or reference sample, the length of the captured polynucleotide fragment is lower.

It is further preferred that the captured polynucleotide fragment as obtained by the method for obtaining a captured polynucleotide fragment according to the present invention comprises at least one gene, or a multitude of genes, such as at least 1, 2, 3, 4, 5, 6, 7, 8, 9, 10, 20, 30, 40, 50, or any number of genes.

In another embodiment, the captured polynucleotide fragment comprises at least one genetic cluster, such as at least one genetic cluster comprising a multitude of genes of at least one (partial) metabolic pathway, preferably wherein said at least one genetic cluster comprises 2, 3, 4, 5, 6, 7, 8, 9, 10, 20, 30, 40, 50, or any number of genes. The person of skill is aware that genes, e.g. bacterial genes, of one (partial) metabolic pathway preferably cluster together. The person of skill also knows methods for analyzing said genetic clusters (operons).

Accordingly, the above method for obtaining a captured polynucleotide fragment according to the first aspect of the present invention provides a technique that allows a cheap and easy way to synthesize a multitude of genes or even entire genetic clusters, such as a genetic cluster comprising genes of one (partial) metabolic pathway.

The sample as provided in step a) of the above method for obtaining a captured polynucleotide fragment according to the first aspect of the present invention can be any kind of sample. In a preferred embodiment, the sample is a sample derived from the environment, such as from soil or from seawater, from a cell-free system, from at least one cell, from at least one virus, from a fossil sample which comprises ancient DNA (i.e. a preserved paleontologic fossil sample), and/or from at least one organism such as from at least one bacterium, fungus, protist, algae, plant, and at least one animal, such as a mammal, for example a human, or a mixture thereof. Most preferably, the sample comprises a mixture of polynucleotide molecules derived from different organisms, such as a sample derived from soil or seawater that comprises a mixture of polynucleotide molecules derived from different organisms. Therefore, an advantage of the present invention is that any kind of polynucleotide molecule can be captured in form of a captured polynucleotide fragment, and subsequently be analyzed. In some embodiments, it is not necessary that the origin of the nucleotide fragment is known. This allows discovering novel molecules, such as novel enzymes, and even novel pathways.

The sample can also be a sample comprising biological fluid (e.g., blood, cerebrospinal fluid, urine, plasma, serum), tissue biopsy, and the like. In some embodiments, the sample is a tissue sample, such as a tumor tissue sample, and may be fresh, frozen, or archival paraffin embedded tissue. The sample can be a sample derived from a human, and can comprise at least one circulating tumor cell.

In a further embodiment, the sample comprises between 10⁻¹⁶ g and 1 g of the polynucleotide molecule, preferably between 10⁻¹⁴ g and 10⁻³ g, more preferably between 10⁻¹² g and 10⁻⁶ g, even more preferably between 10⁻¹¹ g and 10⁻⁹ g, and most preferably between 10⁻¹⁰ g and 2×10⁻¹⁰ g of the polynucleotide molecule. It is one advantage of the present invention that the method for obtaining a captured polynucleotide fragment efficiently works even if only around 100 picogram of a particular polynucleotide is present in the sample.

The polynucleotide fragment that is present in the sample and/or optionally isolated in step b) of the method of the first aspect of the present invention can be an unknown or a known polynucleotide fragment, i.e. a previously uncharacterized or previously characterized polynucleotide fragment and/or molecule. Accordingly, the method of the first aspect of this invention is useful for obtaining a captured polynucleotide fragment, wherein the captured polynucleotide fragment was previously not known, i.e. a novel and previously not described polynucleotide fragment. Alternatively, and/or additionally, the method of the first aspect of this invention is also useful for obtaining a captured polynucleotide fragment, wherein the polynucleotide fragment is a known, i.e. previously disclosed and/or characterized polynucleotide fragment.

The methods of the first aspect of this invention, or of any of the other aspects of the present invention, can be an ex-vivo or in-vitro method.

A further aspect of this invention then relates to a captured polynucleotide fragment, obtained by a method according to the first aspect of the present invention.

Another aspect of the present invention then pertains to a method for obtaining a rearranged captured polynucleotide fragment, comprising:
a) Providing a captured polynucleotide fragment, and
b) Cloning and/or assembling the captured polynucleotide fragment, wherein the cloning and/or assembling involves at least one of:
   (i) Synthetic Chromosome Rearrangement and Modification by LoxP-mediated Evolution (SCRaMbLE),
   (ii) Golden gate cloning,
   (iii) Gibson assembly,
   (iv) Aqua cloning, or
   (v) Any other method for cloning and/or assembly of a polynucleotide molecule,
      thereby obtaining the rearranged captured polynucleotide fragment.

One advantage of the present invention is that the method for obtaining a captured polynucleotide fragment according to the present invention can be combined with any method for genetic rearrangements. One example for genetic rearrangement that is suitable to be combined with the present invention is Synthetic Chromosome Rearrangement and Modification by LoxP-mediated Evolution (SCRaMbLE) according to US 2019/0382776 A1. SCRaMbLE does not comprise a tagmentation step to capture DNA, i.e. no transposase is required for SCRaMbLE.

SCRaMbLE is also not suitable to capture larger DNA fragments that may be present in low abundance within a sample (e.g. metabolic pathway clusters and/or operons). Instead, SCRaMbLE makes use of CRE recombination to genetically rearrange DNA.

Yet a further aspect of this invention then relates to a captured polynucleotide fragment, obtained by a method for obtaining a rearranged captured polynucleotide fragment.

Another aspect then relates to a kit for performing a method according to the first and/or second aspect of the present invention, the kit comprising:
(i) at least one acceptor molecule;
(ii) at least one enzyme, and (iii) at least one transposase, mutant transposase or variant transposase, wherein the transposase, mutant transposase or variant transposase, is bound to at least two adaptors, and wherein each adaptor comprises at least one capture sequence tag (CST).

A further aspect relates to a vector, comprising a captured polynucleotide fragment

according to the present invention, optionally wherein the vector is an expression vector, a bacterial artificial chromosome (BAC), or a yeast artificial chromosome (YAC).

The term “expression vector” means any double-stranded DNA designed to transcribe a nucleic acid molecule, e.g., a construct that contains at least one promoter operably linked to a downstream gene or coding region of interest (e.g., a cDNA or genomic DNA fragment that encodes a protein, or any RNA of interest). Transfection or transformation of the expression construct into a recipient cell allows the cell to express RNA or protein encoded by the expression construct. An expression construct may be a genetically engineered plasmid, bacteriophage or virus genome, or a bacterial or an artificial DNA, such as a yeast artificial chromosome (BAC or YAC), or the like. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct. Further, the term expression construct or vector is intended to also include instances wherein the cell utilized for the assay already endogenously comprises such DNA sequence.

The term “encoding” or more simply “coding” refers to the ability of a nucleotide sequence to code for one or more amino acids. The term does not require a start or stop codon. An amino acid sequence can be encoded in any one of six different reading frames provided by a polynucleotide sequence and its complement. An amino acid sequence can be encoded by desoxyribonucleic acid (DNA), ribonucleic acid (RNA), or artificially synthesized polymers similar to DNA or RNA.

Another aspect of the invention provides a vector, comprising the polynucleotide molecule of the invention. In a preferred embodiment, the vector comprising the polynucleotide molecule is an expression vector, comprising a promoter sequence operably linked to the nucleic acid as defined above.

A “vector” may be any agent that is able to deliver or maintain a polynucleotide molecule in a host cell and includes, for example, but is not limited to, plasmids (e.g., DNA plasmids), naked nucleic acids, viral vectors, viruses, nucleic acids complexed with one or more polypeptide or other molecules, as well as nucleic acids immobilized onto solid phase particles. Vectors are described in detail below. A vector can be useful as an agent for delivering or maintaining an exogenous gene and/or protein in a host cell. A vector may be capable of transducing, transfecting, or transforming a cell, thereby causing the cell to replicate or express nucleic acids and/or proteins other than those native to the cell or in a manner not native to the cell. The target cell may be a cell maintained under cell culture conditions or in other in vivo embodiments, being part of a living organism. A vector may include materials to aid in achieving entry of a nucleic acid into the cell, such as a viral particle, liposome, protein coating, or the like. Any method of transferring a nucleic acid into the cell may be used; unless otherwise indicated, the term vector does not imply any particular method of delivering a nucleic acid into a cell or imply that any particular cell type is the subject of transduction. The present invention is not limited to any specific vector for delivery or maintenance of any nucleic acid of the invention, including, e.g., a polynucleotide fragment of the invention.

In another aspect there is also provided a recombinant cell comprising a captured polynucleotide fragment, and/or a vector as described herein. A “recombinant cell”, sometimes also referred to as “host cell”, is any cell that is susceptible to transformation with a nucleic acid. Preferably, the recombinant or host cell of the invention is a bacterial cell, a yeast cell, a plant cell, an insect cell or a mammalian cell. A preferred recombinant cell is selected from a cell suitable for recombinant expression of the polynucleotide fragment of the invention.

An additional aspect of the present invention relates to a polynucleotide fragment library, comprising at least one captured polynucleotide fragment according to the present invention, preferably comprising at least 5 captured polynucleotide fragment(s), more preferably comprising at least 10 captured polynucleotide fragment(s), more preferably comprising at least 10³, even more preferably comprising at least 10⁴, and most preferably comprising at least 10⁵ captured polynucleotide fragment(s).

Yet another aspect pertains to a polynucleotide fragment library, obtainable by repeating the method according to the first and/or second aspect of this invention at least twice, preferably a multitude of times, such as three times, four times, five times, six times, seven times, eight times, nine times, ten times, twenty times, thirty times, forty times, or repeating the method any other number, and storing the obtained captured polynucleotide fragment(s) in form of a polynucleotide fragment library.

A further aspect relates to a method for identification of at least one compound, at least one protein, and/or at least one secondary metabolite having biological activity, comprising:
a) Providing at least one captured polynucleotide fragment, at least one vector, at least one recombinant cell, or at least one polynucleotide fragment library according to the present invention;
b) Optionally, amplifying the at least one captured polynucleotide fragment, the at least one vector, and/or the at least one recombinant cell;
c) Optionally, transforming the at least one captured polynucleotide fragment and/or the at least one vector into at least one host organism and/or host cell, or transcribing the at least one captured polynucleotide fragment and/or the at least one vector in-vitro;
d) Translating the at least one captured polynucleotide fragment into at least one candidate compound, at least one protein, and/or at least one secondary metabolite, and
e) Screening the at least one candidate compound, the at least one protein, and/or the at least one secondary metabolite for its biological activity.

In one embodiment, the methods of the present invention can be used to identify at least one protein, e.g. at least one enzyme, which then catalyzes the formation of secondary metabolites, and/or secondary compounds that can be tested for their biological activity. Accordingly, the present methods are not restricted to identifying a protein and/or compound having biological activity itself, but can also enable identifying a protein and/or compound that produces at least one protein and/or at least one compound as a secondary metabolite, and/or via at least one intermediary molecule.

In one embodiment, the present invention can be used to identify at least one enzyme, which then produces a compound that can be tested for its biological activity.

In a preferred embodiment, the biological activity of the at least one compound, at least one protein, and/or the at least one secondary metabolite is antibiotic, enzymatic, colorimetric, fluorescent, antibacterial, antifungal, antiviral, anti-parasitic, anti-inflammatory, anti-angiogenic, pro-apoptotic, anti-apoptotic, anti-cancerous, anti-allergic, antimicrobial, anti-aging, analgesic, neuromodulatory, gene expression modifying, gene expression enhancing, gene expression inhibiting, immune-stimulating, immune-modulating, immune-inhibiting, inhibitory, stimulating, enhancing, or any other biological activity.

In yet another preferred embodiment, the at least one host organism and/or host cell is any organism and/or cell that is susceptible to transformation with a nucleic acid. Preferably, the host organism and/or host cell of the invention is a bacterial cell, a yeast cell, a plant cell, a fungal cell, or an animal cell, such as an insect cell or a mammalian cell. A host organism and/or host cell is preferably a cell suitable for recombinant expression of the polynucleotide fragment of the invention.

In a particularly preferred embodiment, the screening of step e) involves a readout indicative for a biological activity, such as a colorimetric, fluorimetric, and/or a spectrophotometric detection method, and/or optionally involves FACS sorting. Alternatively, colony properties can be used as measurement for the screening of step e), such as colony size, number and quantification of zones around colonies that indicate substrate utilization or the killing of other cells.

In another embodiment, the method for identification of at least one compound and/or at least one protein having biological activity further comprises the step:
f) Sequencing of the at least one captured polynucleotide fragment.

In a further embodiment, the enzymatic activity is plastic degrading, cellulolytic, amylolytic, dextranolytic, chitinolytic, proteolytic, esterolytic, depolymerolytic, and/or lipolytic.

In yet another embodiment, the screening for an enzymatic activity comprises transforming the at least one captured polynucleotide fragment and/or the at least one vector into at least one host organism and/or host cell, and growing said at least one host organism and/or host cell in presence of a single and/or a limited source of nutrients, such as on a source comprising a non-biodegradable polymer, cellulose, amylose, dextran, chitin, starch, and/or a milk protein as sole carbon source.

In a particularly preferred embodiment, the non-biodegradable polymer is selected from the group consisting of polyolefins, polyvinyl chloride (PVC), polystyrene (PS), polyethylene (PE), polypropylene (PP), polyethylene terephthalate (PET), polycarbonate (PC), ethylene vinyl alcohol (EVOH), poly lactic acid (PLA), polyethylene terephthalate (PET), polybutylene terephthalate (PBT), polytrimethylene terephthalate (PTT), polyethylene isosorbide terephthalate (PEIT), polyethylene furanoate (PEF), polyamide (PA), polyamide-6, Poly(ε-caprolactam), polycaproamide, polyamide-6,6, Poly(hexamethylene adipamide), Poly(11-aminoundecanoamide) (PA11), polydodecanolactam (PA12), poly(tetramethylene adipamide) (PA4,6), poly(pentamethylene sebacamide) (PA5,10), polyhexamethylene nonanediamideaamide (PA6,9), poly(hexamethylene sebacamide) (PA6,10), poly(hexamethylene dodecanoamide) (PA6,12), poly(m-xylylene adipamide) (PAMXD6), polyhexamethylene adipamide/polyhexamethyleneterephtalamide copolymer (PA66/6T), polyhexamethylene adipamide/polyhexamethyleneisophtalamide copolymer (PA66/6I), polyurethane (PU), acrylonitrile butadiene styrene (ABS), poly(oxide phenylene) (PPO), copolymer of phosphono and carboxylic acid (PCA), high molecular weight polyacrylate, polymethacrylate methyle (PMMA), polyoxymethylene (POM), styrene acrylonitrile (SAN), polyester polymer alloy (PEPA), polyethylene naphthalate (PEN), styrene-butadiene (SB), and blends/mixtures thereof.

In general, there are no limits to the nature of the at least one candidate compound, the at least one protein, and/or the at least one secondary metabolite. In a particularly preferred embodiment, the candidate compound can be a small molecular compound (“small molecule”), a polypeptide, a peptide, a glycoprotein, a peptidomimetic, an antigen binding construct (for example, an antibody, antibody-like molecule or other antigen binding derivative, or an antigen binding fragment thereof), a nucleic acid, such as a DNA or RNA, for example an antisense or inhibitory DNA or RNA, a ribozyme, an RNA or DNA aptamer, siRNA, shRNA, microRNA and the like, including variants or derivatives thereof, a genetic construct for targeted gene editing, such as a CRISPR/Cas9 construct, a guide nucleic acid (gRNA or gDNA), crRNA and/or a tracrRNA.

The term “small molecule”, as used herein, refers to an organic or inorganic molecule, either synthesized or found in nature. A “small molecule” generally has a molecular weight equal to or less than 1000 Da (1 kDa), however the definition of a small molecule is in some embodiments not limited by this number.

The screening method of the invention is preferably performed in a non-human animal system, ex-vivo, or in-vitro. With respect to ex-vivo uses, the method is preferably done in cell free systems or, alternatively, in cell culture. In context of the latter, animal or plant cells are preferably used, such as insect cells.

A further aspect of this invention, which can be combined with any of the other preferred embodiments and/or aspects of the invention, relates to a method for the production of a pharmaceutical composition, the method comprising identifying at least one compound, at least one protein, and/or at least one secondary metabolite having biological activity as above, and formulating the at least one compound, the at least one protein, and/or the at least one secondary metabolite as a pharmaceutical composition, together with a pharmaceutically acceptable carrier and/or excipient.

Another aspect of this invention, which can be combined with any of the other preferred embodiments and/or aspects of the invention, pertains to the use of the at least one compound, the at least one protein, and/or at least one secondary metabolite identified according to the method for identification of at least one compound, at least one protein, and/or at least one secondary metabolite having biological activity, as defined above, for the production of a medicament for use in the prevention and/or treatment of a disease.

Another aspect of the present invention relates to a transposase, mutant transposase or variant transposase, wherein said transposase, mutant transposase or variant transposase, is bound to at least two transposon adaptors, and wherein each adaptor comprises at least one capture sequence tag (CST).

Yet another aspect pertains to the use of a transposase, mutant transposase or variant transposase according to the present invention, for obtaining a captured polynucleotide fragment.

Yet another particularly preferred embodiment relates to the above method for identification of at least one compound, at least one protein, and/or at least one secondary metabolite having biological activity, wherein the at least one candidate compound, at least one protein, and/or at least one secondary metabolite as identified as a compound, protein, and/or secondary metabolite to have biological activity is suitable for the prevention and/or treatment of the disease.

A further aspect pertains to a pharmaceutical composition comprising a captured polynucleotide fragment, a vector, a recombinant cell, a transposase, mutant transposase or variant transposase, and/or the at least one candidate compound, at least one protein, and/or at least one secondary metabolite as identified as a compound, protein, and/or secondary metabolite having biological activity, according to the present invention, together with a pharmaceutically acceptable carrier, stabilizer and/or excipient.

As used herein the term “pharmaceutically acceptable carrier” shall include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Supplementary agents can also be incorporated into the compositions.

The pharmaceutical composition of the invention is formulated to be compatible with its intended route of administration. In a particularly preferred embodiment examples of routes of administration of the pharmaceutical and/or compound of this invention include intravenous, vaginal, oral, intranasal, intrathecal, intra-arterial, intradermal, subcutaneous, transdermal (topical), intracerebroventricular, intraparenchymal, intratumoral, transmucosal, rectal, bronchial, parenteral administration, and any other clinically/medically accepted method for administration of a pharmaceutical and/or a compound.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; anti-bacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride, mannitol or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, mannitol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

The term “intrathecal” as used herein, means introduced into or occurring in the space under the arachnoid membrane which covers the brain and spinal cord. The term “intracerebroventricular” refers to administration of a composition into the ventricular system of the brain, e.g., via injection, infusion, or implantation (for example, into a ventricle of the brain).

As used herein, the term “intraparenchymal” can refer to an administration directly to brain tissue. In other instances, intraparenchymal administration may be directed to any brain region where delivery of one or more compounds of the invention is effective to mitigate or prevent one or more of disorders as described herein.

Sterile injectable solutions can be prepared by incorporating the active compound (e.g., a compound of the invention) in the required amount in an appropriate solvent with one or a combination of ingredients enumerated above, as required, followed by filtered sterilization. Generally, dispersions are prepared by incorporating the active compound of the invention into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Yet another aspect pertains to a compound for use in the treatment of a disease, the compound being selected from a captured polynucleotide fragment, a vector, a recombinant cell, a transposase, mutant transposase or variant transposase, the at least one candidate compound, at least one protein, and/or at least one secondary metabolite as identified as a compound, protein, and/or secondary metabolite having biological activity, or a pharmaceutical composition according to the present invention.

In the following, the at least one captured polynucleotide fragment, the vector, the recombinant cell, the transposase, mutant transposase or variant transposase, and/or the at least one candidate compound, at least one protein, and/or at least one secondary metabolite as identified as a compound, protein, and/or secondary metabolite having biological activity, as well as any pharmaceutical compositions thereof, will be referred to generally as “compounds of the invention”.

According to the present invention, any disease, condition or disorder can be prevented and/or treated by the compound as identified and/or characterized according to the method of this invention. In a preferred embodiment, said disease is a proliferative disease, such as cancer, diabetes, an infectious disease, a metabolic disease, an immune-related disease, a degenerative disease, such as a neurodegenerative disease, for example Alzheimer's disease, and/or aging.

The term “cancer” and “cancer cells” refers to any cells that exhibit uncontrolled growth in a tissue or organ of a multicellular organism. The term “cancer” is understood to mean any cancer or cancerous lesion associated with a certain tissue or tissue cells and can include precursors to the particular cancer disease, for example, atypical ductal hyperplasia or non-atypical hyperplasia. Cancer can be understood as a disease in which a primary tumor or multiple individual primary tumors exist, e.g. in the breast or breasts in case of breast cancer. The term “tumor” refers to an abnormal benign or malignant mass of tissue that is not inflammatory and possesses no physiological function.

Compositions and methods of the present invention may be used to effectively treat individuals suffering from a disease, such as cancer, or to prevent the onset of a particular disease.

As used herein, the term “prevention” shall refer to preventing or delaying the onset of a clinically evident disease, such as a proliferative disease, like cancer, in a subject. The term “treatment”, as used herein, refers to amelioration of one or more symptoms, in particular a partial or total inhibition and/or reduction of symptoms, associated with the disease, such as a proliferative disease, like cancer, in a subject, prevention or delay of the onset of one or more symptoms of the disease, and/or lessening of the severity or frequency of one or more symptoms of the disease. The term “treatment” shall also refer to a partial or total destruction of, for example, diseased cells, or cancer cells. In the context of the present invention, prevention and/or treatment shall include both preventive and/or actual treatment of the disease symptoms of a disease, such as a proliferative disease, like cancer, which can be alleviated and/or even completely removed using said treatment.

In some embodiments, treatment refers to an increased survival (e.g. an increased survival time). For example, treatment can result in an increased life expectancy of a patient. In some embodiments, treatment according to the present invention results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with similar disease without treatment. In some embodiments, treatment according to the present invention results in an increased life expectancy of a patient by more than about 6 month, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with a similar disease without treatment. In some embodiments, treatment according to the present invention results in long term survival of a patient. As used herein, the term “long term survival” refers to a survival time or life expectancy longer than about 40 years, 45 years, 50 years, 55 years, 60 years, or longer.

The terms, “improve,” “increase” or “reduce,” as used herein, indicate values that are relative to a control. In some embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with the same form of disease, who is about the same age and/or gender as the individual being treated.

A further aspect of this invention relates to a compound for use in the prevention and/or treatment of a disease, the compound being a compound according to the present invention.

Yet another aspect of this invention, which can be combined with any of the other aspects or specific embodiments of this invention, relates to a method of preventing and/or treating a disease, condition or disorder in a patient, comprising the administration to the subject a therapeutically effective amount of a compound according to the present invention. As used herein, the term “subject” or “patient” refers to any animal (e.g., a mammal), including, but not limited to, humans, non-human primates, rodents, and the like. Typically, the terms “subject” and “patient” are used interchangeably herein in reference to a human subject. As used herein, the term “subject suspected of having a disease” refers to a subject that presents one or more symptoms indicative of a particular disease, such as cancer (e.g., a noticeable lump or mass in the case of cancer). A subject suspected of having a particular disease may also have one or more risk factors. The term a “subject suspected of having a disease” also encompasses an individual who has received an initial diagnosis (e.g., a CT scan showing a mass) but for whom the sub-type or stage of the particular disease, such as a certain type of cancer, is not known. In some embodiments, the subject is an individual who has been recently been diagnosed with the disease. Typically, early treatment (treatment commencing as soon as possible after diagnosis) is important to minimize the effects of the disease and to maximize the benefits of treatment.

A treatment according to the invention preferably comprises the administration of a therapeutically effective amount of the compound of the invention to a subject in need of the treatment. The term “effective amount” as used herein refers to an amount of a compound that produces a desired effect. For example, a population of cells may be contacted with an effective amount of a compound to study its effect in vitro (e.g., cell culture) or to produce a desired therapeutic effect ex vivo or in vitro. An effective amount of a compound may be used to produce a therapeutic effect in a subject, such as preventing and/or treating a target condition, alleviating symptoms associated with the condition, or producing a desired physiological effect. In such a case, the effective amount of a compound is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the compound (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the compound is administered alone or in combination with another compound, drug, therapy or other therapeutic method or modality. One skilled in the clinical and pharmacological arts will be able to determine an effective amount or therapeutically effective amount through routine experimentation, namely by monitoring a cell's or subject's response to administration of a compound and adjusting the dosage accordingly.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. The pharmaceutical compositions can be included in a container, pack, or dispenser, together with instructions for administration.

For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, serum albumin, polyorthoesters, polylactic acid, poly(butyl cyanoacrylate), and poly(lactic-co-glycolic) acid. Methods for preparation of such formulations will be apparent to those skilled in the art.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

An additionally preferred embodiment of the invention pertains to the above methods of treatment, wherein said treatment comprises administration to said subject a compound as screened according to the method for identifying and/or characterizing a compound suitable for the prevention and/or treatment of a disease, as defined above.

Another aspect of this invention, which can be combined with any of the other aspects or specific embodiments of this invention, relates to the use of a compound according to this invention, for the manufacture of a medicament for the prevention and/or treatment of a disease, condition or disorder.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site).

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES

The figures show:

FIG. 1 is a schematic scheme of the methods for obtaining a captured polynucleotide fragment according to the present invention. In step 1, a (Tn5) transposase loaded with CST adaptor sequences is added to input DNA, e.g. DNA isolated from a metagenomic sample, which leads to the fragmentation of the input DNA and its simultaneous tagging with CST adaptor sequences (tagmentation). Different genes of the input DNA are highlighted in different shades on a black-and-white scale. In step 2, CST-tagged DNA fragments are integrated into an acceptor DNA molecule, such as a plasmid, BAC or YAC. This integration can be performed via different methods according to the present invention and are enabled by properties of the CST adaptor sequence tags (illustrated in FIG. 2). In step 3, acceptor DNA with captured foreign DNA fragments are transformed into a heterologous host. According to the present invention, some methods for fragment integration in step 2 take place in vivo by using the cells' DNA repair machinery (e.g. integration via homology directed repair), and in these cases step 2 and 3 are coupled. The resulting transformants (cells that took up acceptor DNA) can readily be screened for any bioactivity with existing assays, as shown in step 4.

FIG. 2 is a schematic scheme demonstrating different options for DNA capture. After tagmentation, the CST-tagged DNA fragments can be integrated into an acceptor DNA by different methods, including homology-based methods (Option 1), restriction coupled to ligation (Option 2) and recombination (Option 3). Independent of the applied DNA fragment integration method, TransCap enables the capture of foreign DNA into a defined acceptor DNA and cells transformed with these can be directly employed for bioactivity screens with existing assays. DNA capture is thus customisable, and can be adapted e.g. depending on properties of the heterologous host organism (e.g. making use of the highly efficient homology-directed repair in budding yeast) or the downstream application (e.g. making use of restriction sites, or performing further rearrangements using recombination).

FIG. 3 depicts a bioanalyzer trace file showing the shift in size distribution of tagmented DNA (outlined area) using different tagmentation conditions. A shows Tn5 loaded with loxPSym or sequencing adapters, from around 300 bp using a 1:1000 dilution. B shows Tn5 loaded with loxPSym or sequencing adapters, from around 600 bp using a 1:10000 dilution. A linearized 5000 bp plasmid served as input for tagmentation in A and B.

FIG. 4 shows tagmentation of genomic DNA allows capture of tunably-sized large DNA fragments with custom adapters. FemtoPulse traces show the size distribution. (A) High molecular weight genomic DNA extracted with the Nanobind CBB Big DNA kit (Circulomics) from BY4743 and 573 yeast strains. (B) Scaling-up tagmentation reactions from 4 ng to 500 ng input DNA with sequencing adapters produces longer DNA fragments. Varying the dilution of Tn5 (1:10, 1:100, and 1:1000) can tune the length of both high (C) and low (D) input DNA tagmented with sequencing (C) and loxPsym (D) adapters. Tagmented DNA can be successfully amplified with sequencing primers (E) and loxPsym primers (F), while maintaining distinctions in DNA fragment length due to DNA input (E) or Tn5 dilution (F). Tn5 was used for loading custom adapters.

FIG. 5 depicts Oxford nanopore sequencing of captured yeast genomic DNA. Tagmented yeast genomic DNA samples, with and without PCR amplification, were pooled and sequenced on an Oxford Nanopore Flongle flow cell via Ligation Sequencing (SQK-LSK109). Reads were aligned to the yeast genome using minimap2. An exemplary alignment for a region of chromosome X is shown on the IGV genome browser. The top track shows reads with size 3 kb and longer, the bottom track shows all aligned reads independent of size. Watson and crick strand alignments are shown. Gene annotations are shown in the bottom track.

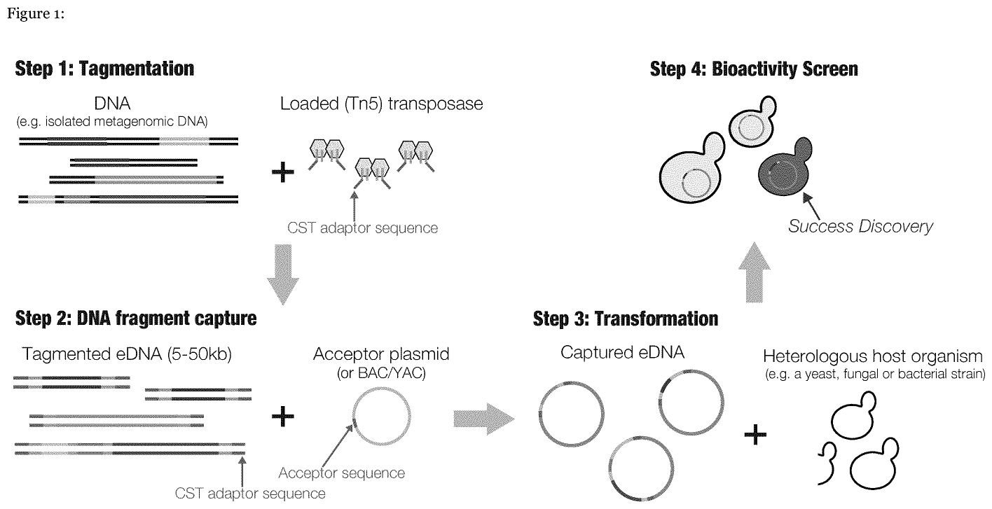

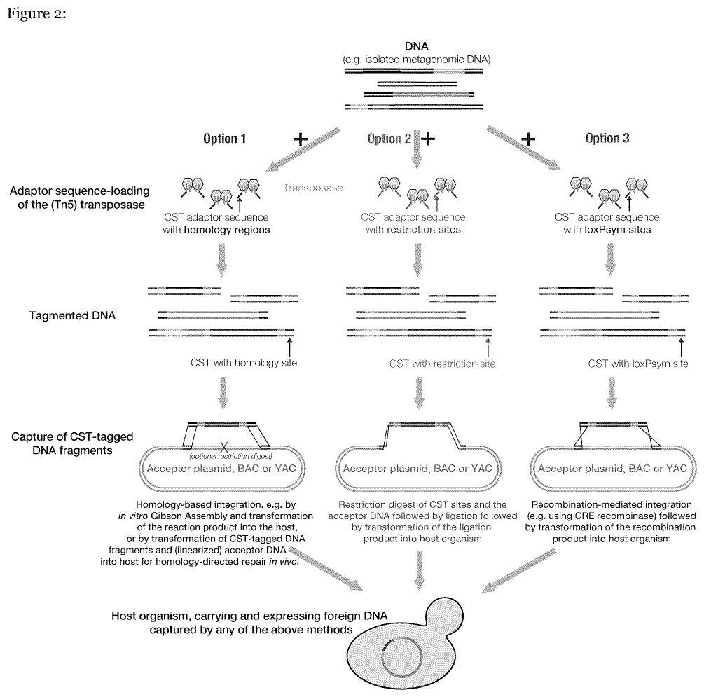

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### Example 1: Captured Polynucleotide Fragments Using Tagmentation

The inventors used the novel methods for obtaining a captured polynucleotide fragment by tagmentation of environmental DNA (eDNA) using Tn5 transposases comprising at least one CST (e.g. a loxPsym sequence), recombination of tagmented eDNA into an acceptor plasmid (e.g. a BAC or YAC), and transformation of captured eDNA into a yeast host strain. The captured eDNA is then screened for a compound, protein, and/or secondary metabolite having a bioactivity, such as an antibiotic, enzymatic, colorimetric, fluorescent, antibacterial, antifungal, antiviral, anti-parasitic, anti-inflammatory, anti-angiogenic, pro-apoptotic, anti-apoptotic, anti-cancerous, anti-allergic, antimicrobial, anti-aging, analgesic, neuromodulatory, gene expression modifying, gene expression enhancing, gene expression inhibiting, immune-stimulating, immune-modulating, immune-inhibiting, inhibitory, stimulating, enhancing, or any other biological activity.

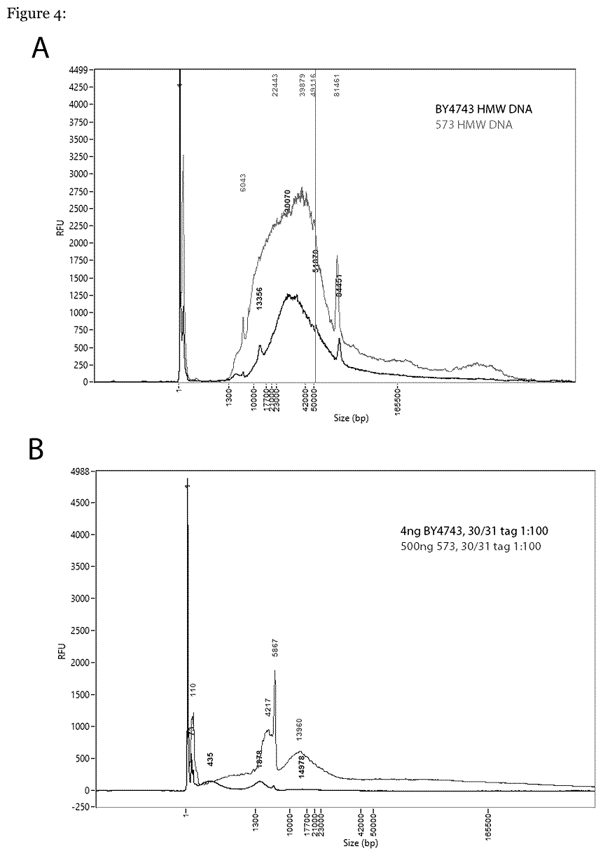

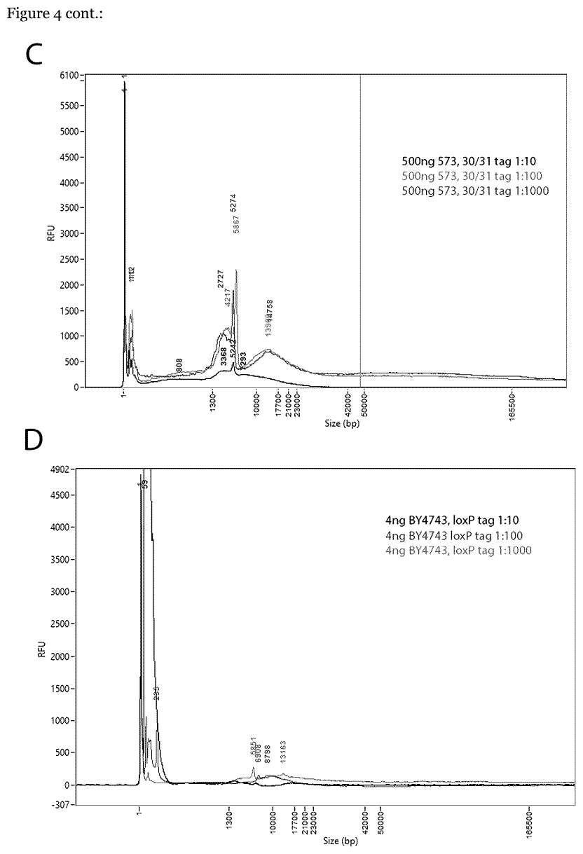

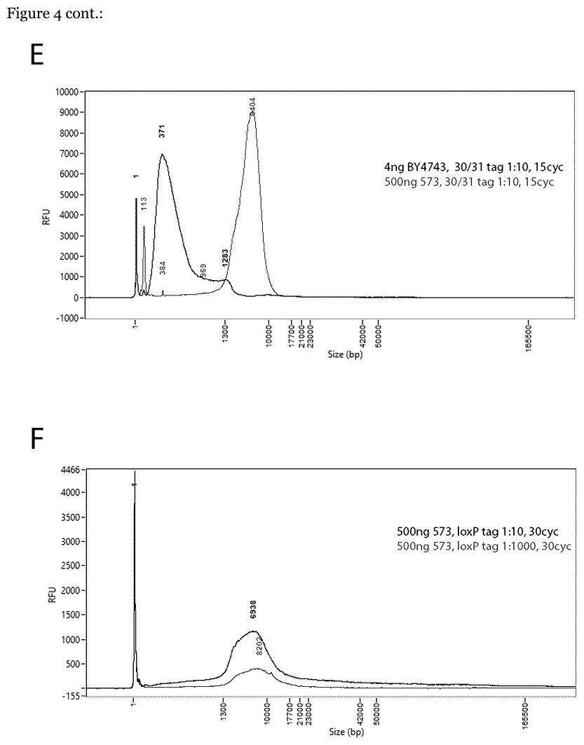

FIG. 4 shows that genomic DNA isolated from two yeast strains can be efficiently tagmented using the present invention. FIG. 4 C-F shows that the present invention is not dependent on the adapters used. Tagmentation works with standard Illumina “30/31” Tn5 adapters (FIG. 4 C; E), as well as with custom “loxP” adapters that have a loxPsym site and a restriction site (FIG. D; F).

Interestingly, the inventors observed that the length of the captured polynucleotide fragment depends on the ratio of the at least one transposase, mutant transposase or variant transposase, to the at least one polynucleotide molecule in the sample, and/or the duration of the contacting in step c). In particular, the length of the captured polynucleotide fragment can be (i) increased by decreasing the ratio of the at least one transposase, mutant transposase or variant transposase, to the at least one polynucleotide molecule in the sample, or (ii) decreased by increasing the ratio of the at least one transposase, mutant transposase or variant transposase, to the at least one polynucleotide molecule in the sample.

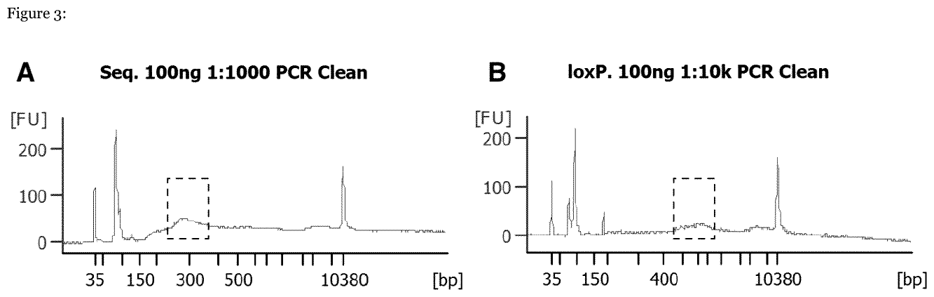

For validation experiments, the inventors synthesized polynucleotide fragments using the methods of the present invention varying in length. FIG. 3 depicts a bioanalyzer trace file showing the shift in size distribution of tagmented DNA (outlined area) using Tn5 loaded with loxPSym or sequencing adapters, from around 300 bp using a 1:1000 dilution (A) to around 600 bp using a 1:10000 dilution (B), using a 5000 bp DNA as input.

Importantly, the present invention can be used to achieve longer fragment size with greater DNA input (that can also be tuned by the transposase to DNA ratios).

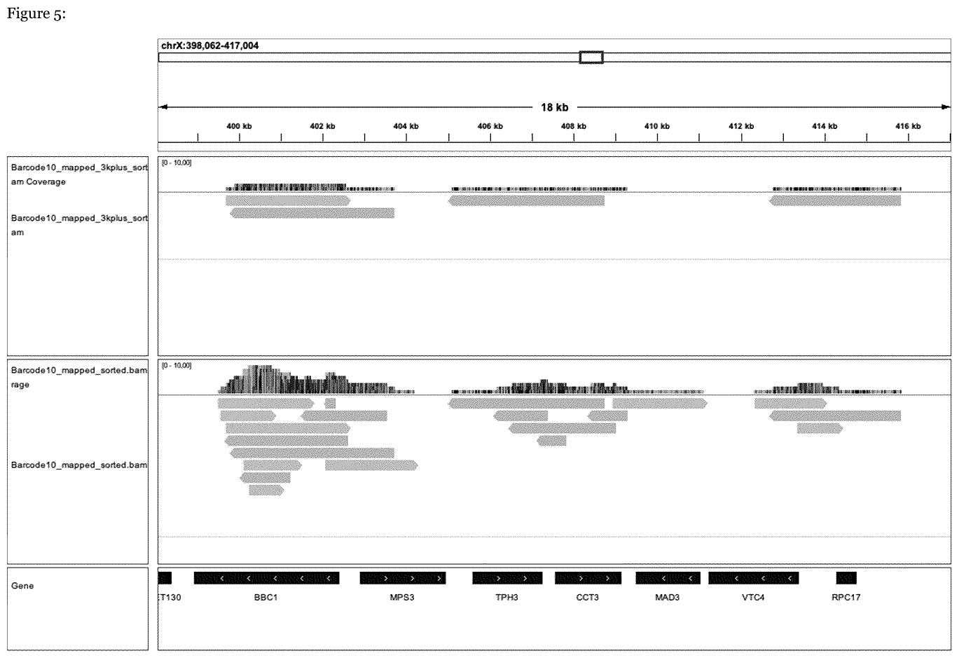

The inventors further confirmed that it is possible to scale up the tagmentation reaction from 5 ng DNA input (this is already scaled up compared to the ˜150 pg DNA concentrations that are generally used for tagmentation reactions of cDNA in single cell sequencing) to 500 ng input. This high amount of DNA allows to sequence whole DNA fragments using Oxford NanoPore sequencing (FIG. 5). Interestingly, multiple genes can be present in a single fragment.

PCR can be used to amplify fragments and maintain the fragment size, which can also be achieved for the custom loxPsym adapters which are amplified with custom primers. Any sequence can additionally be added to the fragments with further PCRs. In general, it is possible to perform the entire tagmentation workflow with standard 30/31 primers, and add further sequences for capture (CSTs, such as loxPsym sites, restriction sites, homology regions) at the PCR step.

### Example 2: Screening for Colorimetric Activity or Fluorescent Activity

Next, the inventors tested whether the method for identification of at least one compound, at least one protein, and/or at least one secondary metabolite having biological activity of the present invention could be used to identify a compound, protein, and/or secondary metabolite having colorimetric activity (pigment) or fluorescent activity.

The inventors performed experiments by transforming cells with captured polynucleotide molecules according to the present invention. Compounds, proteins, and/or secondary metabolites that are, comprise, and/or produce pigments or fluorophores can be identified with FACS sorting or by plating cells, growing colonies, and identifying colonies that are colored or emit fluorescent light. The colorimetric or fluorescence intensity can also be quantified by a well-plate readout, such as by readout of 12-well plates, 24-well plates, 96-well plates, 1284-well plates, or any other multi-well plate. This method allows identifying and characterizing novel colorimetric molecules (pigments) or fluorescent molecules, which can subsequently be purified and used for biological assays.

### Example 3: Screening for Antibiotic Activity

To explore antibiotic functions of the at least one compound, at least one protein, and/or at least one secondary metabolite to be screened, transformed cells can be plated on a bacterial or fungal lawn. In case antibiotics are produced, these can be spotted on the lawn of microbes, and antibiotic activity can be scored by measuring the size of halos resulting from antibiotic activity. Screening procedures are further enhanced through the use of shuttle domains in the plasmids containing the captured DNA, allowing for direct transfer between fungal and bacterial hosts depending on the needs. This enables assaying of antibacterial compounds in a fungal host, and assaying of antifungal activity in a bacterial host. For example, streptomyces colony plates on a Streptococcus pneumoniae bacteria lawn can be analyzed, where clear areas indicate so-called “kill zones” of an antibiotic-producing colony against the pathogenic target (lawn bacterium). The methods of this invention are particularly useful for screening compounds from uncultivable microbes (e.g. from soil), which can allow the discovery of novel compounds to kill or modulate target organisms.

### Example 4: Screening for Enzymatic Activity

Next, the inventors analyzed whether the method for identification of at least one compound, at least one protein, and/or at least one secondary metabolite having biological activity according to the present invention could be used to identify a compound, protein, and/or secondary metabolite having an enzymatic activity. The enzymatic activity may be plastic degrading, cellulolytic, amylolytic, dextranolytic, chitinolytic, proteolytic, esterolytic, depolymerolytic, lipolytic, or any other enzymatic activity.

Here, transformed cells that produce (and secrete) a particular enzyme can be selected in a growth assay that imposes an absolute requirement for this enzymatic activity. This can be done by limiting an essential nutrient, such as a carbon source, to only one single type. If e.g. a cellulose or starch polymer is provided as sole carbon source, cells need to produce a cellulase or amylase to survive and thrive, respectively. Similarly, a poly-ethylene polymer can be provided as sole carbon source to select for cells that produce plastic-degrading enzymes. A large variety of existing assays are available that cover a broad range of enzyme classes (i.e. lipases, proteases, esterases, depolymerases etc.), which, in addition to the cell growth selection, typically also allow a colorimetric or spectrophotometric detection of the respective enzyme activity that is quantitative and can be directly compared between samples and positive and negative controls. The person of skill is well aware of such assays.

As an example, a non-biodegradable polymer can be provided as sole carbon source to select for cells that produce plastic-degrading enzymes, wherein the non-biodegradable polymer can be, for example, a polyolefin, polyvinyl chloride (PVC), polystyrene (PS), polyethylene (PE), polypropylene (PP), polyethylene terephthalate (PET), polycarbonate (PC), ethylene vinyl alcohol (EVOH), poly lactic acid (PLA), polyethylene terephthalate (PET), polybutylene terephthalate (PBT), polytrimethylene terephthalate (PTT), polyethylene isosorbide terephthalate (PEIT), blends/mixtures thereof.

1. A method for obtaining a captured polynucleotide fragment, the method comprising the steps of:
a) Providing a sample, an acceptor molecule, at least one enzyme, and at least one transposase, mutant transposase or variant transposase, wherein the at least one transposase, mutant transposase or variant transposase is bound to at least two adaptors, and wherein each adaptor comprises at least one capture sequence tag (CST);
b) Optionally, isolating at least one polynucleotide molecule from the sample;
c) Contacting the sample and/or the isolated polynucleotide molecule with the at least one transposase, mutant transposase or variant transposase, thereby generating a polynucleotide fragment, wherein at least one, preferably both ends, of the polynucleotide fragment is tagged with the at least one CST, and
d) Contacting the polynucleotide fragment and/or the acceptor molecule with the at least one enzyme, thereby inserting the polynucleotide fragment into the acceptor molecule to obtain the captured polynucleotide fragment.

2. The method according to claim 1, wherein the at least one polynucleotide molecule, the polynucleotide fragment, and/or the captured polynucleotide fragment is a functional or non-functional DNA polynucleotide molecule, such as a single-stranded or double-stranded DNA polynucleotide molecule, or a fragment or derivative thereof, for example a cDNA molecule, a DNA aptamer, and/or a protein-coding or non-coding DNA.

3. The method according to claim 1, wherein the transposase is a DDE transposase, a Tn5 transposase, a Tn3 transposase, a Tn7 transposase, a Tn10 transposase, a Tn552 transposase, a Tn903 transposase, a sleeping beauty transposase, a Mu transposase, a MuA transposase, Mos1, Hermes, ProtoRAG, a HUH-like (Y1-/Y2-) transposase, a Y-transposase and/or a S-transposase, such as Tn1549, a Vibhar transposase, such as a Vibhar transposase from Vibrio harveyi, and/or a mutant or variant thereof, preferably wherein the transposase is Tn5 transposase, or a mutant or variant thereof.

4. The method according to any one of claim 1, wherein the acceptor molecule is a DNA polynucleotide molecule, such as a single-stranded or double-stranded DNA polynucleotide molecule, or a fragment or derivative thereof, preferably wherein the acceptor molecule is a DNA polynucleotide molecule selected from a plasmid, a bacterial artificial chromosome (BAC), a yeast artificial chromosome (YAC), viral DNA, and/or genomic DNA.

5. The method according to any one of claim 1, wherein the at least one capture sequence tag (CST) is a lox recombination sequence, such as a loxP sequence, preferably a symmetric loxP (symLoxP) sequence, a rox recombination sequence, a restriction enzyme recognition sequence, such as a Typal sequence, and/or a homology sequence, wherein the acceptor molecule comprises at least one acceptor nucleic acid sequence, such as an acceptor loxP sequence, preferably an acceptor symLoxP sequence, an acceptor Rox sequence, a restriction enzyme recognition sequence, for example a TypeII sequence, and/or a homology sequence, wherein the at least one enzyme is Cre recombinase, Dre recombinase, a restriction enzyme, a ligase, and/or a homology-directed repair (HDR) enzyme, and wherein the inserting of step d) occurs by recombination, restriction and ligation, and/or homology-directed repair (HDR).

6. The method according to any one of claim 1, wherein the length of the captured polynucleotide fragment depends on the ratio of the at least one transposase, mutant transposase or variant transposase, to the at least one polynucleotide molecule in the sample, and/or the duration of the contacting in step c), optionally wherein the length of the captured polynucleotide fragment can be (i) increased by decreasing the ratio of the at least one transposase, mutant transposase or variant transposase, to the at least one polynucleotide molecule in the sample, or (ii) decreased by increasing the ratio of the at least one transposase, mutant transposase or variant transposase, to the at least one polynucleotide molecule in the sample.

7. The method according to any one of claim 1, wherein the sample is a sample derived from the environment, such as from soil or from seawater, from a cell-free system, from at least one cell, from at least one virus, from a fossil sample which comprises ancient DNA (i.e. a preserved paleontologic fossil sample), and/or from at least one organism such as from at least one bacterium, fungus, protist, algae, plant, and animal, such as a mammal, for example a human, or a mixture thereof, optionally wherein the sample comprises between 10⁻¹⁶ g and 1 g of the polynucleotide molecule, preferably between 10⁻¹⁴ g and 10⁻³ g, more preferably between 10⁻¹² g and 10⁻⁶ g, even more preferably between 10⁻¹¹ g and 10⁻⁹ g, and most preferably between 10⁻¹⁰ g and 2×10⁻¹⁰ g of the polynucleotide molecule.

8. A captured polynucleotide fragment, obtained by a method according to claim 1.

9. A method for obtaining a rearranged captured polynucleotide fragment, comprising:
a) Providing a captured polynucleotide fragment obtained by a method according to claim 1, and
b) Cloning and/or assembling the captured polynucleotide fragment, wherein the cloning and/or assembling involves at least one of:
(i) Synthetic Chromosome Rearrangement and Modification by LoxP-mediated Evolution (SCRaMbLE),
(ii) Golden gate cloning,
(iii) Gibson assembly,
(iv) Aqua cloning, or
(v) Any other method for cloning and/or assembly of a polynucleotide molecule,
thereby obtaining the rearranged captured polynucleotide fragment.

10. A kit for performing a method according to claim 1, the kit comprising:
(i) at least one acceptor molecule;
(ii) at least one enzyme, and
(iii) at least one transposase, mutant transposase or variant transposase, wherein the transposase, mutant transposase or variant transposase, is bound to at least two adaptors, and wherein each adaptor comprises at least one capture sequence tag (CST).

11. A vector, comprising a captured polynucleotide fragment obtained by the method of claim 1, optionally wherein the vector is an expression vector, a bacterial artificial chromosome (BAC), or a yeast artificial chromosome (YAC), or a recombinant cell comprising the vector or the captured polynucleotide fragment.

12. A polynucleotide fragment library, comprising at least one captured polynucleotide fragment obtained by the method of claim 1, preferably comprising at least 5 captured polynucleotide fragment(s), more preferably comprising at least 10⁵ captured polynucleotide fragment(s), more preferably comprising at least 10³, even more preferably comprising at least 10⁴, and most preferably comprising at least 10⁵ captured polynucleotide fragment(s), optionally wherein the polynucleotide fragment library is obtainable by repeating the method according to any claim 1, at least twice, preferably a multitude of times, such as three times, four times, five times, six times, seven times, eight times, nine times, ten times, twenty times, thirty times, forty times, or repeating the method any other number, and storing the obtained captured polynucleotide fragment(s) in form of a polynucleotide fragment library.

13. A method for identification of at least one compound, at least one protein, and/or at least one secondary metabolite having biological activity, comprising:
a) Providing at least one captured polynucleotide fragment obtained by the method of claim 1, at least one vector comprising the at least one captured polynucleotide fragment, at least one recombinant cell comprising the at least one captured polynucleotide fragment, or at least one polynucleotide fragment library comprising the at least one captured polynucleotide fragment;
b) Optionally, amplifying the at least one captured polynucleotide fragment, the at least one vector, and/or the at least one recombinant cell;
c) Optionally, transforming the at least one captured polynucleotide fragment and/or the at least one vector into at least one host organism and/or host cell, or transcribing the at least one captured polynucleotide fragment and/or the at least one vector in-vitro;
d) Translating the at least one captured polynucleotide fragment into at least one candidate compound, at least one protein, and/or at least one secondary metabolite, and
e) Screening the at least one candidate compound, the at least one protein, and/or the at least one secondary metabolite, for its biological activity, optionally wherein said screening involves a readout indicative for said biological activity, such as a colorimetric, fluorimetric, and/or a spectrophotometric detection method.

14. The method according to claim 13, wherein said biological activity is antibiotic, enzymatic, colorimetric, fluorescent, antibacterial, antifungal, antiviral, anti-parasitic, anti-inflammatory, anti-angiogenic, pro-apoptotic, anti-apoptotic, anti-cancerous, anti-allergic, antimicrobial, anti-aging, analgesic, neuromodulatory, gene expression modifying, gene expression enhancing, gene expression inhibiting, immune-stimulating, immune-modulating, immune-inhibiting, inhibitory, stimulating, enhancing, or any other biological activity, optionally wherein the enzymatic activity is polymer degrading, cellulolytic, amylolytic, dextranolytic, chitinolytic, proteolytic, esterolytic, depolymerolytic, and/or lipolytic.

15. The method according to claim 14, wherein the polymer is selected from the group consisting of polyolefins, polyvinyl chloride (PVC), polystyrene (PS), polyethylene (PE), polypropylene (PP), polyethylene terephthalate (PET), polycarbonate (PC), ethylene vinyl alcohol (EVOH), poly lactic acid (PLA), polyethylene terephthalate (PET), polybutylene terephthalate (PBT), polytrimethylene terephthalate (PTT), polyethylene isosorbide terephthalate (PEIT), polyethylene furanoate (PEF), polyamide (PA), polyamide-6, Poly(ε-caprolactam), polycaproamide, polyamide-6,6, Poly(hexamethylene adipamide), Poly(11-aminoundecanoamide) (PA11), polydodecanolactam (PA12), poly(tetramethylene adipamide) (PA4,6), poly(pentamethylene sebacamide) (PA5,10), polyhexamethylene nonanediamideaamide (PA6,9), poly(hexamethylene sebacamide) (PA6,10), poly(hexamethylene dodecanoamide) (PA6,12), poly(m-xylylene adipamide) (PAMXD6), polyhexamethylene adipamide/polyhexamethyleneterephtalamide copolymer (PA66/6T), polyhexamethylene adipamide/polyhexamethyleneisophtalamide copolymer (PA66/6I), polyurethane (PU), acrylonitrile butadiene styrene (ABS), poly(oxide phenylene) (PPO), copolymer of phosphono and carboxylic acid (PCA), high molecular weight polyacrylate, polymethacrylate methyle (PMMA), polyoxymethylene (POM), styrene acrylonitrile (SAN), polyester polymer alloy (PEPA), polyethylene naphthalate (PEN), styrene-butadiene (SB), and blends/mixtures thereof.
